(19) United States
(12) Patent Application Publication
(10) Pub. No.: US 2021/0303725 A1
(43) Pub. Date: Sep. 30, 2021

(54) Partially customized machine learning models for data de-identification

(71) Applicant: Google LLC, Mountain View, CA (US)

(72) Inventors: Tzvika Hartman, Mazkeret Batyz (IL); ltay Laish, Timrat (IL); Jiyang Chen, Waterloo (CA); Kaveh Ketabchi, Markham (CA); Gavin Bee, Kitchener (CA); Rohit Talreja, Santa Clara, CA (US); Yossi Matias, Tel Aviv (IL); Andrew Max, San Francisco, CA (US)

(21) Appl. No.: 17/216,891

(22) Filed: Mar. 30, 2021

Related U.S. Application Data
(60) Provisional application No. 63/001,655, filed on Mar. 30, 2020.

(51) Int. Cl. G06F 21/62; G16H 10/60; G06N 3/08; G06K 9/62

(52) U.S. Cl. CPC G06F 21/6245; G06K 9/6259; G06N 3/08; G16H 10/60

(57) ABSTRACT

Apparatus and methods related to de-identifying data are provided. An example method includes receiving, by a computing device, input data comprising text. The method further includes applying a neural network to a tokenized representation of the input text, to generate an embedding based on contextual information associated with an entity. The method also includes predicting, by the neural network and based on the embedding, whether the input data comprises protected data in the text, wherein the neural network has been trained on a training dataset that has been partially customized based on the entity. The method further includes de-identifying the protected data in the text upon a determination that the input data comprises protected data in the text.

### CROSS REFERENCE TO RELATED APPLICATIONS

This application claims priority to U.S. Provisional Patent Application No. 63/001,655, filed on Mar. 30, 2020, which is hereby incorporated by reference in its entirety.

### BACKGROUND

This disclosure relates to a method of developing a system or machine learning model that is used for de-identifying data, such as, for example, protected data to be edited, modified, redacted, encrypted, and/or otherwise de-identified. For example, protected data such as protected health information within a dataset, including unstructured free text notes, radiology images containing identifying information such as patient name, and the like, may be de-identified.

For example, in a healthcare setting, a de-identified clinical dataset may be created by locating words and/or phrases that may be used to identify an individual from records in the dataset, and replacing such words and/or phrases with surrogate data or context-specific labels. For example, a text note that says: “John London complains of chest pain that started on Jan. 1 2012” may be de-identified as “[Person Name] complains of chest pain that started on [Date]”. It may be desirable for the de-identification process to have high recall (i.e., sensitivity) to prevent a public release of text containing protected data. Also, for example, it may be desirable for the de-identification process to have reasonable precision, because unnecessarily removal of non-identifying text may limit a usefulness of a dataset (e.g., to researchers in building and training machine learning models based on de-identified datasets).

### SUMMARY

In this document, a method is described for generating or developing a de-identification system, based on pre-trained machine learning models, that may be partially customized and designed for a specific dataset, e.g., that of a particular organization, industry segment, provider of insurance, healthcare, legal services, and so forth. Fully customized systems may exhibit high performance, but generally require a large corpus of labeled training examples, which may be costly to build, and may require an inordinate amount of time to generate. However, a partially customized system can be configured to have performance commensurate with a fully customized system, but may be achieved more easily, and at a relatively lower cost.

Several ways of constructing a partially customized de-identification system are described herein, including (1) with a relatively small set of labeled data and (2) without labeled data.

In one aspect, a computer-implemented method is provided. The method includes receiving, by a computing device, input data comprising text. The method further includes applying a neural network to a tokenized representation of the input text, to generate an embedding based on contextual information associated with an entity. The method also includes predicting, by the neural network and based on the embedding, whether the input data comprises protected data in the text, wherein the neural network has been trained on a training dataset that has been partially customized based on the entity. The method further includes, upon a determination that the input data comprises protected data in the text, de-identifying the protected data in the text.

In another aspect, a server is provided. The server includes one or more processors. The server additionally includes memory storing computer-executable instructions that, when executed by the one or more processors, cause the server to perform operations. The operations include receiving, by a computing device, input data comprising text. The operations further include applying a neural network to a tokenized representation of the input text, to generate an embedding based on contextual information associated with an entity. The operations also include predicting, by the neural network and based on the embedding, whether the input data comprises protected data in the text, wherein the neural network has been trained on a training dataset that has been partially customized based on the entity. The operations further include, upon a determination that the input data comprises protected data in the text, de-identifying the protected data in the text.

In another aspect, an article of manufacture is provided. The article of manufacture includes one or more computer readable media having computer-readable instructions stored thereon that, when executed by one or more processors of a computing device, cause the computing device to carry out operations. The operations include receiving, by a computing device, input data comprising text. The operations further include applying a neural network to a tokenized representation of the input text, to generate an embedding based on contextual information associated with an entity. The operations also include predicting, by the neural network and based on the embedding, whether the input data comprises protected data in the text, wherein the neural network has been trained on a training dataset that has been partially customized based on the entity. The operations further include, upon a determination that the input data comprises protected data in the text, de-identifying the protected data in the text.

In another aspect, a computing device is provided. The computing device includes one or more processors and data storage. The data storage has stored thereon computer-executable instructions that, when executed by one or more processors, cause the computing device to carry out operations. The operations include receiving, by the computing device, input data comprising text. The operations further include applying a neural network to a tokenized representation of the input text, to generate an embedding based on contextual information associated with an entity. The operations also include predicting, by the neural network and based on the embedding, whether the input data comprises protected data in the text, wherein the neural network has been trained on a training dataset that has been partially customized based on the entity. The operations further include, upon a determination that the input data comprises protected data in the text, de-identifying the protected data in the text.

In another aspect, a system is provided. The system includes means for receiving, by a computing device, input data comprising text; means for applying a neural network to a tokenized representation of the input text, to generate an embedding based on contextual information associated with an entity; means for predicting, by the neural network and based on the embedding, whether the input data comprises protected data in the text, wherein the neural network has been trained on a training dataset that has been partially customized based on the entity; and upon a determination that the input data comprises protected data in the text, means for de-identifying the protected data in the text.

The foregoing summary is illustrative only and is not intended to be in any way limiting. In addition to the illustrative aspects, embodiments, and features described above, further aspects, embodiments, and features will become apparent by reference to the figures and the following detailed description and the accompanying drawings.

### BRIEF DESCRIPTION OF THE FIGURES

FIG. 1 illustrates an example architecture for a partially customized neural network for de-identifying data, in accordance with example embodiments.

FIG. 2 depicts a table with results of dataset analysis for a fully customized neural network for de-identifying data, in accordance with example embodiments.

FIG. 3 depicts another table with results of dataset analysis for an off-the-shelf neural network for de-identifying data, in accordance with example embodiments.

FIGS. 4A-C are example graphical representations of system performance for a partially customized neural network for de-identifying data, in accordance with example embodiments.

FIG. 5 depicts another table with results of dataset analysis for a neural network with a customized token embedding, in accordance with example embodiments.

FIG. 6 is a diagram illustrating training and inference phases of a machine learning model, in accordance with example embodiments.

FIG. 7 depicts a distributed computing architecture, in accordance with example embodiments.

FIG. 8 is a block diagram of a computing device, in accordance with example embodiments.

FIG. 9 depicts a network of computing clusters arranged as a cloud-based server system, in accordance with example embodiments.

FIG. 10 is a flowchart of a method, in accordance with example embodiments.

### DETAILED DESCRIPTION

In an increasingly electronic world, there are many instances where certain types of data may need to be de-identified, obfuscated, deleted, encrypted, and/or otherwise transformed, so as to limit its unnecessary distribution. In certain industries such as the healthcare industry, financial services industry, information technology services industry, and so forth, protection of information is desirable, and can sometimes be mandated by various laws. For example, the Health Insurance Portability and Accountability Act of 1996 (HIPAA) establishes national standards in the United States for the protection of an individual's medical records. Also, for example, the General Data Protection Regulation (GDPR) provides a framework for the collection and processing of personal information from individuals living in the European Union. Due to a large volume of such records, a vast number of types of data to be potentially protected, and a dynamic nature of the legal and/or ethical frameworks, it may sometimes be difficult to adequately identify data to be protected, and/or protect such data.

An automated de-identification system may not be able to guarantee performance on all types of text that it may process. In some instances, data in structured data, such as forms, may be easy to process and de-identify. For example, known data fields for names, dates, social security numbers, credit card information, and so forth may be de-identified. However, unstructured text, such as those is clinical notes (e.g., medical discharge summaries) may be more challenging to process, since the notes may vary by note purpose, institutional conventions, individual physicians, medical diagnoses. Accordingly, such unstructured text may include protected data in ways that may be challenging to identify and protect.

In some instances, such a challenge may be addressed by customizing the de-identification system. For example, a machine learning system can be customized to learn about the formatting and jargon used in a particular setting (e.g., an organization). For example, a note may include text such as, “John referred to Alzheimer's clinic . . . ”. An off-the-shelf system may recognize “Alzheimer' s” as a medical condition and de-identify the text to “[PersonNameTag] referred to Alzheimer's clinic . . . ”. However, a system customized on a target organization's labeled data may recognize that a “Dr. Alzheimer” and his clinic appear within the target organization, and such a customized system may then de-identify the text to “[PersonNameTag] referred to [PersonNameTag]'s clinic . . . ”. Generally, one model customized on a particular entity can remove “Alzheimer” based on knowledge of an existence of a physician in the particular entity with the name “Alzheimer”, whereas another model might fail to identify “Dr. Alzheimer” as protected data and not de-identify the text, thereby disclosing the physician's identity.

Fully customized systems are generally expensive to deploy because a sufficiently large number of labeled examples have to be generated, often by human annotators, for training a machine learning model to perform automated de-identification. However, as described herein, a partially customized system may be trained to achieve performance levels that may be comparable to a fully customized system.

Accordingly, partial system customization with labeled data is described. For example, an organization implementing such a method may have the resources to provide labeled data (e.g., data that is tailored to the organization). Since labeled data is an expensive resource, and may often be generated by human annotators, it may be desirable to determine a number of labeled examples that can improve performance over an off-the-shelf system, and another number of labeled examples that can improve performance to be comparable to a fully customized system.

In another configuration, a partial system customization with unlabeled data may be described. For example, an organization may avoid certain legal and privacy concerns involved with annotating data, and improve performance using a large set of the organization's unlabeled data. In such instances, a custom token embedding (a data representation) can be generated for the machine learning system.

Performance of a fully customized system on three medical datasets may be evaluated for purposes of comparison, and a model may be trained on a portion of a dataset and evaluated on the remainder of the dataset.

Performance of an off-the-shelf system may also be evaluated for purposes of comparison. In such an instance, an organization may not provide labeled data, and instead may use a pre-trained model as-is, i.e., off-the-shelf.

### Introduction and Overview

### Data Sources Used in System Development

The HIPAA de-identification standard specifies use of either an “Expert Determination” or a “Safe Harbor” method to de-identify data. In the Safe Harbor method, 18 types of patient PHI are removed (Name, Address, Day & Month, Age over 89, Telephone, etc.). Publicly available datasets of de-identified clinical records meeting the Safe Harbor criteria are utilized herein. These datasets have been de-identified by replacing PHI with plausible but realistic surrogate information. The systems described herein are evaluated on this surrogate PHI. In this document, where applicable, the term PHI may be used to refer to such surrogate PHI.

Other datasets used may include the i2b2-2006 and i2b2-2014 datasets from the i2b2 National Center for Biomedical Computing for the NLP Shared Tasks Challenges. The i2b2-2006 de-identification guidelines conform to the Safe Harbor standard and further add “hospital name” and “doctor name” to the list of removed identifiers. The i2b2-2014 guidelines further add “year” to the list of removed identifiers. These datasets were hand labeled and surrogated prior to their release in the public domain.

Another dataset used may be the PhysioNet gold standard corpus of de-identified medical text, containing surrogate information. The labels in this dataset were generated by annotators based on the i2b2-2014 guidelines.

Another dataset used may be the Medical Information Mart for Intensive Care III (MIMIC-III) dataset. This dataset was de-identified before release using the PhysioToolkit de-identification software package, which expands on Safe Harbor to include ethnicities, clinical provider numbers, and other indirect identifiers. The PHI identification process may be based on regular expressions, and may have a substantial number of false positives. Detectable false positives may be substituted with plausible text. The remaining placeholders may be substituted with fictitious values from distributions consistent with the PHI type specified in the placeholder. Three subsets may be generated from the MIMIC-III corpus: mimic3-radiology, mimic3-echo, and mimic3-discharge, each containing a thousand notes of the prescribed type.

### Partial Customization With Labeled Data

In a first embodiment, a de-identification machine learning model is trained primarily on a dataset (dataset “A” in this document, several examples of which are described with particularity), and with some limited amount of labeled data from a second dataset (“B”), which could, for example, be a dataset of a particular organization (e.g., a healthcare company, a financial company) developing the machine learning model. The partially customized system may be configured to have performance metrics in de-identifying protected data at par with a fully customized system. For purposes of this description, a fully customized system may be one that includes a dataset with a large number (e.g., greater than 10,000) of labeled examples for training a new machine learning model. Some possible scenarios for training such a partially customized model are: (a) train using labeled examples from dataset B, (b) pre-train the model on dataset A, and then perform further training on the labeled examples from dataset B, and (c) jointly train the model using a mixture (e.g., an even mixture) of examples from datasets A and B.

Thus, in one embodiment a method is described for developing a data de-identification system. The method includes the steps of (a) obtaining a first dataset (A) of training examples containing a type of protected data (e.g., personally identifiable information (PII), protected health information (PHI), payment card industry (PCI) information, and so forth); (b) creating a limited set of customized labeled training examples containing PHI in a second dataset (B); and (c) training a machine learning model to de-identify protected data using at least the customized labeled training examples from dataset B. Dataset B may be a training dataset that may be partially customized based on an entity. For example, the entity may be an industry segment (e.g., financial, legal, healthcare, higher education, information technology, automotive, airline, and so forth), an organization in an industry segment (e.g., a university, a law firm, a company, a hospital, a clinic, and so forth).

In some embodiments, the training of the neural network may include a pre-training of the neural network based on a first dataset comprising labeled training data, and then, a training of the pre-trained neural network based on labeled data from the training dataset that has been partially customized based on the entity. For example, the training step (c) may involve the steps of first training the machine learning model on dataset A, and subsequently tuning the model by retraining the machine learning model using the customized labeled training examples from dataset B. This process may be sometimes referred to herein as “A then B”.

In some embodiments, the training of the neural network may include training the neural network based on a mixture of (i) labeled data from the training dataset that has been partially customized based on the entity, and (ii) an unlabeled training dataset. For example, the training step (c) may involve using a mixture of dataset A and the customized labeled training examples from dataset B. This process may be sometimes referred to herein as “A mix B”.

In some embodiments, a platform may be provided to generate a manually programmed dictionary of terms indicative of protected data. At least a portion of the training dataset that has been partially customized based on the entity may be received from the platform. In some embodiments, providing of the platform may include providing an applications programming interface (API). For example, a tool or mechanism, such as a cloud-based applications programming interface (API) may be provided to enable a human expert to build a manually programmed dictionary of terms containing protected data (e.g., PII, PHI, PCI) in order to create the limited set of training examples in dataset B.

The dataset B can take a variety of formats, including a radiology dataset, discharge or nursing dataset including free text notes, a transcript of a patient-physician conversation, etc., or a combination thereof. In one embodiment the dataset may be a radiology dataset, and the limited set of customized labeled training examples in dataset B may include at least fifty (50) training examples. Dataset B may take the form of free text unstructured notes, e.g., nursing or discharge notes, and the model may be trained using only dataset B. The limited set of customized labeled training examples may be at least one hundred (100) training examples, and may, for example, include five hundred training examples. Performance of the model may improve with a number of training examples used.

### Partial Customization Without Labeled Data

Another way of partial customization of a machine learning model without using labeled data from an entity is described. In some embodiments, the training of the neural network may include obtaining a first dataset (e.g., dataset A) comprising labeled training data that includes first protected data, where the labeled training data is not based on the entity; obtaining a second dataset comprising unlabeled training data that includes second protected data, where the unlabeled training data is based on the entity; and generating, based on the second dataset, a customized token embedding of the second protected data. For example, the system may be trained on dataset A only, but using a customized token embedding that is generated using a second unlabeled dataset B. Dataset A may include labeled data that may not be specific to an organization, and dataset B may typically be a very large dataset from the organization. A token embedding maps a discrete word to a floating point vector so that vectors corresponding to similar words cluster together, thus providing information about a language model to the machine learning system. Customized token embeddings may be generated using large unlabeled text corpora. In some instances, using domain-specific corpora may improve system performance. Customized token embeddings may be generated using an algorithm to estimate word representations in a vector space (e.g., a word2vec algorithm), including distributed representations of words and phrases and their compositionality, as may be used by neural information processing systems. Generally, the term “word2vec algorithm” as used herein, may refer to a family of architectures and/or optimizations for neural networks that can be configured to learn word embeddings from large datasets.

Accordingly, a method of developing a protected data (e.g., PHI) de-identification system may be described. The method may include steps of: (a) providing a first dataset (e.g., dataset A) of labeled training examples containing protected data, where the labels may not be based on the entity; (b) providing a second dataset (e.g., dataset B) of unlabeled training examples containing protected data, where the unlabeled training examples may be based on the entity; (c) using dataset B to generate a customized token embedding for protected data; (d) applying the token embedding generated from step (c) to dataset A, and (e) training a machine learning model on dataset A using the token embedding of step (d).

In one configuration the customized token embedding may be built using an algorithm to estimate word representations in a vector space (e.g., a word2vec algorithm). In another embodiment, a tool is provided for a human expert to create a customized token for terms containing PHI, for example a cloud-based API.

In some embodiments, the protected data may include protected health information, and the training dataset that has been partially customized based on the entity may include radiology images that include the protected health information. For example, dataset B may take the form of free text unstructured notes, a radiology dataset including radiology images containing protected data (e.g., burned in to the images), or a combination thereof, and the neural network may be trained on dataset B.

In another aspect, a method of creating a custom de-identification model (e.g., by a user such as an administrator), with a preexisting or provided “baseline” de-identification system or model. This may be, for example, an “off-the shelf” system such as a default de-identification system in a healthcare de-identification application programming interface (API). Performance of such a baseline de-identification system may be improved by creating a limited set of customized labeled training examples containing protected data from dataset B, and training a machine learning model to de-identify the protected data using at least the customized labeled training examples from dataset B.

### Network Architecture

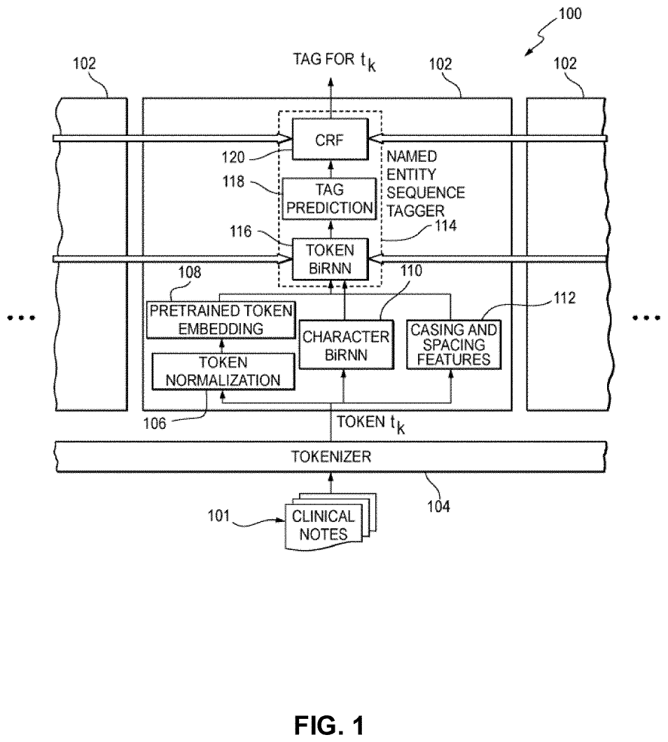

FIG. 1 illustrates an example architecture for neural network 100 for de-identifying data, in accordance with example embodiments. The machine learning model described herein implements de-identification of medical notes and named entity sequence tagging. As described herein, unbiased recall, precision and F1 (harmonic mean of precision and recall) of neural network 100 on the i2b2-1014 dataset is comparable to existing systems (e.g., named-entity recognition based open-source neural networks such as a NeuroNER system).

FIG. 1 depicts a high level system design, with a machine learning de-identification block 102 repeated for each token in the sequence. In some embodiments, neural network 100 may receive input data comprising text. For example, neural network 100 may take as input, clinical notes or other medical record data, containing words (tokens) containing protected health information. Also, for example, neural network 100 may de-identify data that has to be protected. For example, neural network 100 may output a non-identifying tag e.g., [name] for the token, in accordance with example embodiments.

The example architecture for neural network 100 may include the following one or more components.

In some embodiments, neural network 100 may generate the tokenized representation by generating one or more tokens based on the input text. For example, neural network 100 may include a tokenizer 104, to generate one or more tokens from input text 101. In some embodiments, clinical notes from a particular dataset described above (e.g., i2b2-2014, Mimic-III, or PhysioNet) may be transformed to tokens. For example, “Patient prescribed 50 mg . . . ” may be utilized to generate one or more tokens such as, for example, “Patient”, “prescribed”, “50”, “mg”, and so forth.

In some embodiments, neural network 100 may, for each token of the one or more tokens, convert (i) a character to a lowercase letter, and (ii) a numeral to zero. For example, neural network 100 may include a token normalization block 106 that may substitute characters and numerals to standard symbols. For example, token normalization block 106 may convert characters to lowercase letters, and digits to zero.

In some embodiments, the neural network may include a pre-trained token embedding model to map the tokenized representation to a first multidimensional vector space. For example, neural network 100 may include pre-trained token embedding 108 that maps each token of the one or more tokens into a first feature representation space (e.g., a 200-dimensional vector space). In some embodiments, a global vector representation for words may be used (e.g., an unsupervised learning algorithm such as a GloVe representation, or another custom mapping).

In some embodiments, the neural network may include a bi-directional recurrent neural network (BiRNN) to generate a character-based token embedding that maps each token of the one or more tokens to a second multidimensional vector space. For example, BiRNN 110 may generate a corpus-specific, character-based token embedding into a second feature representation space (e.g., a 25-dimensional vector space). In some aspects, such an embedding may augment the token embedding by learning corpora-specific token and sub-token patterns. Such an augmentation may be useful to process unstructured text. For example, out-of-vocabulary words, abbreviations, and/or common prefix/suffix information may be processed based on the embedding into the second feature representation space.

In some embodiments, neural network 100 may include a casing feature 112. Casing feature 112 may provide information about a token's casing (e.g., upper case, lower case, mixed capitalization, etc.), and a number of spaces and/or line breaks preceding a token.

In some embodiments, neural network 100 may include a named entity sequence tagger 114. Named entity sequence tagger 114 may convert a token sequence to a tag sequence, while taking into account context information. For example, in an example sentence “Mr. Jack London was examined”, “London” may be tagged as a person's name. In some embodiments, neural network 100 may include a second BiRNN to add contextual information to the tokenized representation. For example, named entity sequence tagger 114 may include a token BiRNN 116 to add contextual information to an extracted token information.

In some embodiments, neural network 100 may include a prediction layer to project the first multidimensional vector space onto a probability distribution over a plurality of tags, wherein the plurality of tags are indicative of the protected data. For example, named entity sequence tagger 114 may include a tag prediction layer 118 to project the first feature representation space (e.g., the 200-dimensional vector space) onto a probability distribution over a collection of tags. For example, tag prediction layer 118 may project the 200-dimensional vector space onto a probability distribution over a collection of PHI tags, such as, for example, “name”, “age”, “location”, “other”, and so forth, including a “not PHI” tag. In some embodiments, named entity sequence tagger 114 may include a conditional random field 120.

In some embodiments, the neural network can include a second prediction layer based on conditional random field 120, wherein the conditional random field 120 determines whether a tag of the plurality of tags is consistent as a sequence. For example, conditional random field 120 may impose an additional structured prediction layer to determine that labels corresponding to protected data are consistent as a sequence. For example, conditional random field 120 may ensure that PHI labels are consistent as a sequence within a natural language model, and/or within a context of the entity.

In some embodiments, the computing device (e.g., a mobile device) can determine a request to de-identify potentially protected data in a given input data. Then, the computing device (e.g., a mobile device) can send the request to de-identify the potentially protected data from the computing device (e.g., a mobile device) to a second computing device (e.g., a cloud server). The second computing device (e.g., a cloud server) can include a trained version of neural network 100. Subsequently, the computing device (e.g., a mobile device) can receive, from the second computing device (e.g., a cloud server), the predicting of whether the given input data comprises protected data. Then, the computing device (e.g., a mobile device) can de-identify the protected data in the given input data. In some implementations, the computing device (e.g., a mobile device) can receive, from the second computing device (e.g., a cloud server), the de-identified protected data based on the predicting of whether the given input data includes protected data in the text.

In some embodiments, the computing device can obtain a trained neural network. Then, the computing device can de-identify the protected data based on a predicting of whether the input data includes protected data, by using the trained neural network obtained by the computing device.

Neural network 100 can be a fully-convolutional neural network as described herein. During training, neural network 100 can receive as inputs one or more input text. Neural network 100 can include layers of nodes for processing input text. Example layers can include, but are not limited to, input layers, convolutional layers, activation layers, pooling layers, and output layers. Input layers can store input data, such as tags and/or embeddings from other layers of neural network 100. Convolutional layers can compute an output of neurons connected to local regions in the input. In some examples, the predicted outputs can be fed back into neural network 100 again as input to perform iterative refinement. Activation layers can determine whether or not an output of a preceding layer is “activated” or actually provided (e.g., provided to a succeeding layer). Pooling layers can downsample the input. For example, neural network 100 can involve one or more pooling layers to downsample the input by a predetermined factor (e.g., a factor of two) in the horizontal and/or vertical dimensions. Output layers can provide an output of neural network 100 to software and/or hardware interfacing with neural network 100; e.g. to hardware and/or software used to display, print, communicate and/or otherwise provide textual documents and/or images with appropriately de-identified data.

### Training of the Neural Network

Referring again to FIG. 1, in some example embodiments, blocks 110, 116, 118 and 120 may be trained with labeled training examples, block 108 may be trained on large numbers of unlabeled examples, and blocks 106 and 112 may be configured with hardcoded rules.

Neural network 100 may be trained using cross entropy loss over a given set of labeled examples as a loss function, and by applying a gradient descent optimization algorithm. The gradient descent optimization algorithm may be a sub-gradient method, such as an adaptive gradient algorithm (Adagrad). In some instances, a stochastic gradient update function with a batch size of twenty may be used. In some embodiments, a dropout may be applied in layers 1 and 2 of the BiRNNs described herein.

Evaluation results may be determined for recall (as a percent of detected PHI out of total PHI), precision (as a percent of detected PHI which was indeed PHI), and F1 score (a harmonic mean of recall and precision). In some embodiments, a balance between recall and precision may be tuned by setting an activation bias for “not PHI” prior to applying a conditional random field block (e.g., conditional random field 120). In an off-the-shelf system, such a tuning may be achieved using heuristics and/or manual monitoring.

In some embodiments, model development experiments may be performed using a de-identification system with respect to FIG. 1. For example, two partial customized systems, a fully customized system and an off-the-shelf system may be analyzed, as described below.

### Partial Customization With a Small Number of Labeled Training Examples

In some embodiments, a neural network (e.g., neural network 100) may be trained on a first dataset A (e.g., i2b2-2014), augmented with “n” labeled PHI instances from a second dataset B (e.g., a dataset that has been partially customized based on an entity, such as a particular organization, or a publicly available dataset), and subsequently tested on dataset B. For example, dataset B may include labeled examples from the PhysioNet, mimic-radiology, and mimic-discharge datasets. The additional “n” samples from dataset B may, in one embodiment, be the training set for the neural network. For example, the neural network can be trained on these “n” data points from dataset B. For purposes of this description, such a neural network may be referred to as “only B”.

In some embodiments, training the neural network may include additional tuning based on an existing partially trained neural network. For example, a neural network may be pre-trained on dataset A, and subsequently trained on labeled instances from dataset B. For purposes of this description, such a neural network may be referred to as “A then B”.

In some embodiments, the neural network may be jointly trained on datasets A and B. For example, the neural network may be jointly trained using an even mixture of samples from dataset A and dataset B. For purposes of this description, such a neural network may be referred to as “A mix B”.

### Partial Customization With a Large Number of Unlabeled Training Examples

In some embodiments, the neural network may be trained on dataset A only, using a customized token embedding that may be generated using an unlabeled dataset B. Dataset A may comprise labeled data, where the labels may not be specific to an entity, and dataset may be a large set of unlabeled data. A customized token embedding maps a discrete word to a floating point vector. Generally, vectors corresponding to similar words can be clustered together, thus providing information about an underlying natural language model to the neural network. Customized token embeddings may be generated using large unlabeled text corpora. In some examples, using a domain-specific corpora may improve system performance. For example, a GloVe token embedding with 2.2 million unique tokens that can be used in some partial customization scenarios described herein, may be substituted with a customized token embedding built using a word to vector representation (e.g., a word2vec algorithm), using tokens (words) that appear at least a threshold number of times (e.g., ten times). A large training dataset may be generated, for example, an embed-mimic dataset with 2 million notes, and 101,000 unique tokens, as a general medical embedding. Based on embed-mimic dataset, more specific embeddings may be generated using an embed-mimic-nursing dataset that includes 223,000 notes, and 37,000 unique tokens, an embed-mimic-radiology dataset that includes 522,000 notes, and 24,000 unique tokens, and an embed-mimic-discharge dataset that includes 59,000 notes, and 31,000 unique tokens.

### Fully Customized System

Training and testing of the neural network may be performed on dataset A (e.g., i2b2-2014) to design a fully customized system.

### Off-the-Shelf System

Training of the neural network may be performed on dataset A, and testing may be performed on a different dataset B.

### Results: Fully Customized System

For comparison purposes, a fully customized model may be trained, for example, on three datasets that include a threshold number of PHI for a fully trained model.

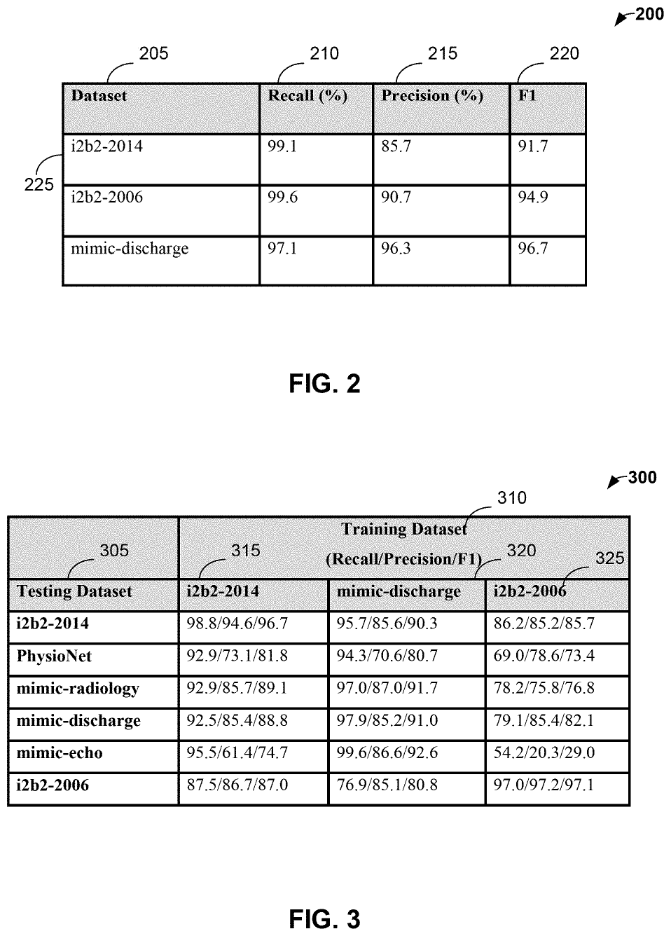

FIG. 2 depicts a table 200 with results of dataset analysis for a fully customized neural network for de-identifying data, in accordance with example embodiments. The results for a fully customized system on three datasets are provided in Table 2, indicating that a Recall percentage for such systems exceeds 97%. First column 205 lists the three datasets that may be used, i2b2-2014, i2b2-2006, and mimic-discharge. Second column 210 provides values for the Recall (as a percentage) for each of the three datasets. Third column 215 provides values for the Precision (as a percentage) for each of the three datasets. Fourth column 220 provides values for F1 for each of the three datasets.

These results may be analyzed to infer some challenges in fully customized de-identification systems. For example, first row 225 provides results for the i2b2-2014 dataset. For example, from the 15,201 PHI elements in the evaluation set, the model based on the i2b2-2014 dataset classified 15,000 true positives, 116 false negatives, and approximately 2,500 false positives. In particular, errors may be analyzed for a specific example of protected data, such as “Name”. For example, de-identification of “Name” corresponded to 14 false negatives, indicating that the protected data corresponding to a name was not predicted 14 times. These included three physician initials, one single-letter suffix for a patient (e.g., “I” as in “John Smith I”), one dataset-mislabeled apostrophe-s, and nine names. All nine names were dictionary words (“. . . saw Guy in PT”, “Strong to cover for . . . ”). Accordingly, contextual information appears not to be incorporated into these fully customized automated de-identification systems.

False positives may remove information that may be useful to researchers. Such false positives are indicative of what may be unnecessarily lost, and may provide intuition for the workings of an algorithm. The false positives corresponding to “Name” also included medical terms that are similar to names (e.g., “thrombosed St. Jude valve”, “chronic indwelling Foley”, “per Bruce Protocol”, “Zoran”, and so forth), which could be corrected using heuristics based on the context (e.g., medical terminology), and/or the entity (e.g., a specific healthcare facility where employees have such names). Also, for example, there may be errors due to an overreliance on sentence structure. For example, based on the sentence structure “[Title] [space] [FirstName] [space] [LastName],” the second word after a title may be erroneously labeled as a “Name”. For example, in the sentence “Ms Mitchel awoke feeling . . . ”, the first word after the title “Ms” is “Mitchel” and the second word after the title is “awoke”. Accordingly, the word “awoke” may be erroneously labeled as a “Name”. As another example, misspellings may create words that are not recognizable in a dictionary for a language model (e.g., “Interested [sic] in quitting smoking”).

### Results: Off-the-Shelf System

In some instances, an organization may deploy a pre-trained off-the-shelf system with no customization. Several models may be tested on example datasets with compatible labeling schemes, and values for recall/precision/F1 for all PHI types may be obtained. A cross-dataset analysis may be performed for the protected data “Name”.

For example, a neural network based on the i2b2-2014 dataset and tested on the PhysioNet dataset yields precision/recall/F1 values of 76.6/60.5/67.6. Error analysis indicates that 272 of the 441 false negatives (i.e. missed PHI) are of type “Location”, and consist mainly of “MICU”, “PMICU”, “cath lab”, and similar labels. Further analysis revealed that these initials appear only in the PhysioNet dataset, but not in the i2b2-2014 dataset, thus providing a good example of an off-the-shelf system that fails to identify entity related information. When “Location” is not included in the analysis, the precision/recall/F1 values improved to 89.1/59.8/71.6. Such an improvement illustrates that a deployment of to de-identification system could consider be based on an off-the-shelf model together with heuristics based on an entity, such as a list of local PHI abbreviations. Such heuristics may, in some example, be obtained from a manual error analysis.

As another example, a neural network based on the mimic-discharge model and tested on the mimic-radiology dataset yields precision/recall/F1 values of 65.7/90.9/76.2. Error analysis illustrates that 595 of the 597 false negatives are of type “Id”. Of these errors, 577 correspond to a 7-digit record number that appears at the top of each note. Accordingly, this error is dataset-specific and may be avoided with heuristics based on an entity. For example, upon inclusion of relevant heuristics, the precision/recall/F1 values improved to 99.4/95.2/97.3; results that are on par with a fully customized model.

Also, for example, a neural network based on the mimic-discharge model and tested on the mimic-echo dataset yields precision/recall/F1 values of 99.7/98.7/99.2. These values are on par with a fully customized model, thereby illustrating that de-identification of some datasets may be accomplished without a fully customized system.

FIG. 3 depicts a table 300 with results of dataset analysis for an off-the-shelf neural network for de-identifying data, in accordance with example embodiments. In particular, table 300 presents results for a full cross-dataset analysis using “Name” only. First column 305 lists a number of testing datasets, such as, i2b2-2014, PhysioNet, mimic-radiology, mimic-discharge, mimic-echo, and i2b2-2006. The corresponding training datasets 310 are the i2b2-2014 dataset 315, mimic-discharge dataset 320, and i2b2-2006 dataset 325. Results show more variability than in a fully customized model, although values for the recall exceed 90%. The exception is for values for the i2b2-2006 dataset 325. Error analysis indicates that made-up names used in the i2b2-2006 dataset contained little information, thereby restricting the model's ability to learn.

### Results: Partial Customization Using a Small Number of Labeled Examples

In some embodiments, we may use the i2b2-2014 dataset as the large labeled dataset A, and we may perform experiments using the PhysioNet, mimic-radiology, and mimic-discharge datasets as examples of a partially labeled dataset B.

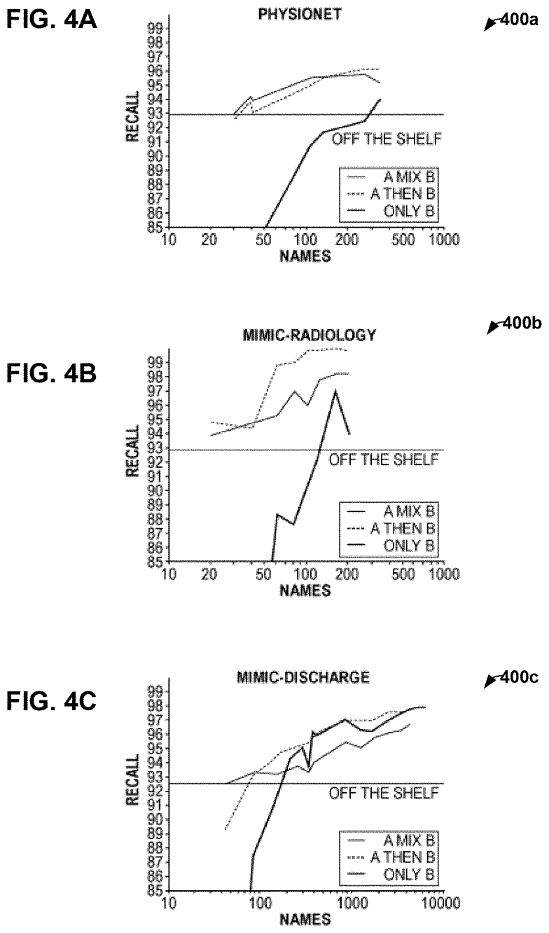

FIGS. 4A-C are example graphical representations of system performance for a partially customized neural network for de-identifying data, in accordance with example embodiments. FIGS. 4A-C are plots 400a, 400b, and 400c, respectively, of system performance as a function of number of labeled names provided for model training. In each of plots 400a, 400b, and 400c, an off-the-shelf system may be trained on dataset A (identified as i2b2-2014, as explained below), and may then be partially customized using labeled examples from a second or target dataset B. Then the de-identification system may be evaluated on dataset B. The performance of a system trained on “only B” examples is provided for comparison. Additionally, the performance of an off-the-shelf system is also shown (as a horizontal line in each subfigure). FIGS. 4A-C show the plots 400a, 400b, and 400c of recall as a function of a number of labels with “Name” for the PhysioNet (as illustrated in FIG. 4A), Mimic-radiology (as illustrated in plot 400b) and Mimi-discharge (as illustrated in plot 400c) as the example datasets for dataset B. The labeled names on the x-axis of plots 400a, 400b, and 400c represent a number of instances of a partially labelled dataset used in model development. As illustrated in these plots, the plot of model training using “A mix B” approximately follows the plot of model training using “A then B”. From the model training “only B” curves for PhysioNet and mimic-radiology, it may be inferred that there may not be an adequate amount of data for the models complete learning. However, the datasets appear to lead to improved performance when they are supplemented from the “A then B” models. For the mimic-radiology dataset, illustrated in plot 400b, using approximately twenty (20) labeled examples in “A then B” model training improved the performance over the corresponding off-the-shelf result described previously, as indicated by the horizontal line. Although this small number (20) may seem surprising, this may be understood based on a relatively uniform structure for radiology notes that may be learned from a context surrounding the examples. For the more varied PhysioNet dataset, as illustrated in plot 400a, approximately a hundred (100) labeled examples may provide similar performance related benefits.

The plot for the larger mimic-discharge dataset, illustrated in plot 400c, illustrates that if over approximately five hundred (500) labels are available, a de-identification system may be trained on dataset B to achieve high performance levels. At about a thousand (1000) labels, performance levels may be comparable to a fully customized system (e.g., 97.1%). Also, for example, at about a thousand (1000) labeled examples, there may be diminishing returns from further efforts in labeling additional examples. Also, for example, at about eighty (80) labels, the “A then B” model may demonstrate better performance than an off-the-shelf system, again demonstrating the usefulness of partially customized systems trained on relatively small labeled datasets.

### Results: Partial Customization Using a Large Number of Unlabeled Examples

In some embodiments, a model may be trained using the i2b2-2014 dataset as dataset A, with different tokenized embeddings, and the test dataset B may be selected from datasets such as PhysioNet, mimic-radiology, mimic-discharge, and mimic-echo. Dataset A may include labeled data that are not entity-specific.

For each choice of dataset B, three different tokenized embeddings may be evaluated. For example, the tokenized embeddings may be based on a generic GloVe embedding, embed-mimic, and the embed-mimic-*. In some evaluations, for the PhysioNet database, which includes nursing data, a subset of the nursing data may be used from a much larger mimic corpus. The embeddings based on embed-mimic, and the embed-mimic-* use a word2vec algorithm. For these mimic datasets, values for recall/precision/F1 on protected data such as “Name” may be obtained. For the PhysioNet database, where the labeling matches the labeling in the i2b2-2014 database, values for recall/precision/F1 on all types of protected data may be obtained.

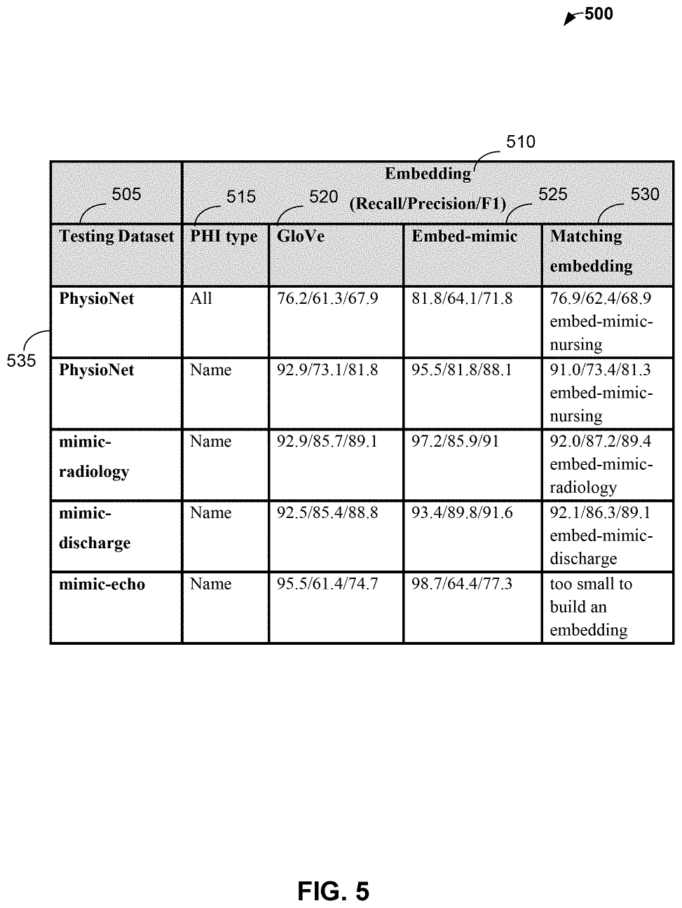

FIG. 5 depicts another table 500 with results of dataset analysis for a neural network with a customized token embedding, in accordance with example embodiments. Table 500 comprises a first column 505 that lists testing datasets such as PhysioNet 535, where an embedding includes all types of PHI. Other testing datasets in first column 505 include a PhysioNet dataset where an embedding includes “Name” as the only type of PHI. Additional testing datasets are mimic-radiology, mimic-discharge, and mimic-echo. Different types of embeddings 510 are shown. Second column 515 lists the type of PHI included in embedding 510. Embeddings 510 may be based on GloVe, with results indicated in third column 520; embed-mimic, with results indicated in fourth column 525, and a matching embedding in fifth column 530.

Table 500 illustrates that switching from a tokenized embedding based on GloVe to a tokenized embedding based on embed-mimic improves results for all datasets. For example, first row 535 indicates for the PhysioNet dataset as the testing dataset, that the values for recall/precision/F1 for the tokenized embedding based on the GloVe dataset are 76.2/61.3/67.9, as provided in third column 520, and these values improve for the tokenized embedding based on the embed mimic database to 81.8/64.7/71.8, as provided in fourth column 525. Also, using a matching embedding resulted in equivalent or decreased performance. For example, as illustrated in first row 535 and fifth column 530, for a matching embedding with the embed-mimic-nursing dataset, the values for recall/precision/F1 are 76.9/62.4/68.9, with performance values that are equivalent to or less than the performance values for the tokenized embedding based on the GloVe dataset (respective values 76.2/61.3/67.9).

Further analysis of the false negatives in the specific embeddings reveals that a large percentage (70-100%) of false positives appear to be from out-of-vocabulary tokens, thereby illustrating that these specific embeddings did not encompass a large enough vocabulary. Thus, an entity (e.g., an organization) may gain improvements from a partial customization technique as described herein. However, performance may be improved when the entity relies on a sufficiently large entity specific corpus.

As described herein, same datasets may be analyzed through various levels of customization, thereby providing a robust view of a level of expected performance of a de-identification system under different scenarios.

As illustrated in FIGS. 4A-C, entities with resources to provide a small amount of labeled data may benefit from partial customization of de-identification systems. Labeling even a small amount of protected data, for example, approximately 20 to 80 examples of PHI, may improve system performance over a performance for an off-the-shelf solution. Labeling approximately a thousand 1000 PHI may give results comparable to fully customized models. In some embodiments, use of customized embeddings using a large set of their unlabeled data, entities may avoid the cost and privacy concerns of labeling data, while gaining in performance over off-the-shelf-systems. Generally, baseline performance may be improved with additional de-identification techniques, such as adding entity-specific or generic heuristics, and/or enhancing a pure machine learning system with a human in the loop.

It will be appreciated that when generating a partially customized machine learning model to perform de-identification, various training scenarios using a limited set of labeled training examples and the embodiments such as “A mix B”, “A then B”, or “only B”, as described herein, can be implemented in the model architecture of FIG. 1 during a model training exercise.

In particular, in the “A mix B” embodiment, BiRNN 110, token BiRNN 116, tag prediction layer 118 and conditional random field 120 may be trained using an even mixture of labeled examples, in this case unlabeled examples from dataset A and labeled examples from dataset B.

In the “A then B” embodiment, BiRNN 110, token BiRNN 116, tag prediction layer 118 and conditional random field 120 of FIG. 1 may be pre-trained on labeled samples from dataset A, and then a subsequent model training exercise may be performed using the labeled examples from dataset B.

In the “only B” embodiment, BiRNN 110, token BiRNN 116, tag prediction layer 118 and conditional random field 120 may only be trained on the labeled examples from dataset B.

To facilitate creating the customized labeled training examples from dataset B for model training, in one possible configuration, a tool may be provided for a human expert to create customized labels, for example, using a cloud-based applications programming interface (API). As another example, when creating customized token for terms containing PHI a cloud-based API could be used.

In another aspect, it is possible for a user (e.g., an administrator) to create a custom de-identification model with a preexisting or provided “baseline” de-identification system, for example an “off-the shelf” system such as a default or existing de-identification system in a healthcare de-identification application programming interface (API). In the context of a protected health information (PHI) de-identification model, a performance of such a baseline de-identification system may be improved by creating a limited set of customized labeled training examples containing PHI from a dataset (e.g., dataset B), and training a machine learning model to de-identify PHI using at least the customized labeled training examples from the dataset B.

### Training Machine Learning Models for Generating Inferences/Predictions

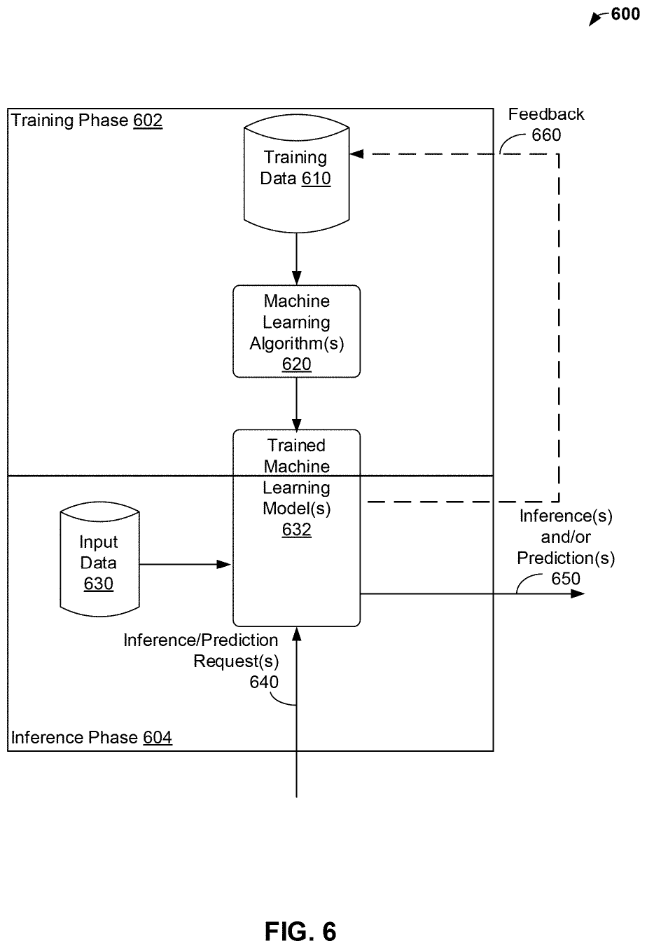

FIG. 6 shows diagram 600 illustrating a training phase 602 and an inference phase 604 of trained machine learning model(s) 632, in accordance with example embodiments. Some machine learning techniques involve training one or more machine learning algorithms, on an input set of training data to recognize patterns in the training data and provide output inferences and/or predictions about (patterns in the) training data. The resulting trained machine learning algorithm can be termed as a trained machine learning model. For example, FIG. 6 shows training phase 602 where one or more machine learning algorithms 620 are being trained on training data 610 to become trained machine learning model 632. Then, during inference phase 604, trained machine learning model 632 can receive input data 630 (e.g., input data comprising text) and one or more inference/prediction requests 640 (perhaps as part of input data 630) and responsively provide as an output one or more inferences and/or predictions 650 (e.g., predict whether the input data comprises protected data in the text, and/or de-identifies the protected data in the text).

As such, trained machine learning model(s) 632 can include one or more models of one or more machine learning algorithms 620. Machine learning algorithm(s) 620 may include, but are not limited to: an artificial neural network (e.g., a herein-described convolutional neural networks, a recurrent neural network, a Bayesian network, a hidden Markov model, a Markov decision process, a logistic regression function, a support vector machine, a suitable statistical machine learning algorithm, and/or a heuristic machine learning system). Machine learning algorithm(s) 620 may be supervised or unsupervised, and may implement any suitable combination of online and offline learning.

In some examples, machine learning algorithm(s) 620 and/or trained machine learning model(s) 632 can be accelerated using on-device coprocessors, such as graphic processing units (GPUs), tensor processing units (TPUs), digital signal processors (DSPs), and/or application specific integrated circuits (ASICs). Such on-device coprocessors can be used to speed up machine learning algorithm(s) 620 and/or trained machine learning model(s) 632. In some examples, trained machine learning model(s) 632 can be trained, reside and execute to provide inferences on a particular computing device, and/or otherwise can make inferences for the particular computing device.

During training phase 602, machine learning algorithm(s) 620 can be trained by providing at least training data 610 as training input using unsupervised, supervised, semi-supervised, and/or reinforcement learning techniques. Unsupervised learning involves providing a portion (or all) of training data 610 to machine learning algorithm(s) 620 and machine learning algorithm(s) 620 determining one or more output inferences based on the provided portion (or all) of training data 610. Supervised learning involves providing a portion of training data 610 to machine learning algorithm(s) 620, with machine learning algorithm(s) 620 determining one or more output inferences based on the provided portion of training data 610, and the output inference(s) are either accepted or corrected based on correct results associated with training data 610. In some examples, supervised learning of machine learning algorithm(s) 620 can be governed by a set of rules and/or a set of labels for the training input, and the set of rules and/or set of labels may be used to correct inferences of machine learning algorithm(s) 620.

Semi-supervised learning involves having correct results for part, but not all, of training data 610. During semi-supervised learning, supervised learning is used for a portion of training data 610 having correct results, and unsupervised learning is used for a portion of training data 610 not having correct results. Reinforcement learning involves machine learning algorithm(s) 620 receiving a reward signal regarding a prior inference, where the reward signal can be a numerical value. During reinforcement learning, machine learning algorithm(s) 620 can output an inference and receive a reward signal in response, where machine learning algorithm(s) 620 are configured to try to maximize the numerical value of the reward signal. In some examples, reinforcement learning also utilizes a value function that provides a numerical value representing an expected total of the numerical values provided by the reward signal over time. In some examples, machine learning algorithm(s) 620 and/or trained machine learning model(s) 632 can be trained using other machine learning techniques, including but not limited to, incremental learning and curriculum learning.

In some examples, machine learning algorithm(s) 620 and/or trained machine learning model(s) 632 can use transfer learning techniques. For example, transfer learning techniques can involve trained machine learning model(s) 632 being pre-trained on one set of data and additionally trained using training data 610. More particularly, machine learning algorithm(s) 620 can be pre-trained on data from one or more computing devices and a resulting trained machine learning model provided to a particular computing device, where the particular computing device is intended to execute the trained machine learning model during inference phase 604. Then, during training phase 602, the pre-trained machine learning model can be additionally trained using training data 610, where training data 610 can be derived from kernel and non-kernel data of the particular computing device. This further training of the machine learning algorithm(s) 620 and/or the pre-trained machine learning model using training data 610 of the particular computing device's data can be performed using either supervised or unsupervised learning. Once machine learning algorithm(s) 620 and/or the pre-trained machine learning model has been trained on at least training data 610, training phase 602 can be completed. The trained resulting machine learning model can be utilized as at least one of trained machine learning model(s) 632.

In particular, once training phase 602 has been completed, trained machine learning model(s) 632 can be provided to a computing device, if not already on the computing device. Inference phase 604 can begin after trained machine learning model(s) 632 are provided to the particular computing device.

During inference phase 604, trained machine learning model(s) 632 can receive input data 630 and generate and output one or more corresponding inferences and/or predictions 650 about input data 630. As such, input data 630 can be used as an input to trained machine learning model(s) 632 for providing corresponding inference(s) and/or prediction(s) 650 to kernel components and non-kernel components. For example, trained machine learning model(s) 632 can generate inference(s) and/or prediction(s) 650 in response to one or more inference/prediction requests 640. In some examples, trained machine learning model(s) 632 can be executed by a portion of other software. For example, trained machine learning model(s) 632 can be executed by an inference or prediction daemon to be readily available to provide inferences and/or predictions upon request. Input data 630 can include data from the particular computing device executing trained machine learning model(s) 632 and/or input data from one or more computing devices other than the particular computing device.

Input data 630 can include structured and/or unstructured text, images with text, and so forth. For example, input data 630 can include medical notes, transcripts, radiology images, and so forth.

Inference(s) and/or prediction(s) 650 can include tokenized embeddings, predictions, pre-trained models, partially trained models, a version of input text with de-identified protected data, and/or other output data produced by trained machine learning model(s) 632 operating on input data 630 (and training data 610). In some examples, trained machine learning model(s) 632 can use output inference(s) and/or prediction(s) 650 as input feedback 660. Trained machine learning model(s) 632 can also rely on past inferences as inputs for generating new inferences.

Neural network 100 can be an example of machine learning algorithm(s) 620. After training, the trained version of network 100 can be an example of trained machine learning model(s) 632. In this approach, an example of inference/prediction request(s) 640 can be a request to de-identify protected data appearing in input text, and a corresponding example of inferences and/or prediction(s) 650 can be an output of a version of the input text with de-identified protected data. In some examples, a given computing device can include the trained neural network 100, perhaps after training of the neural network. Then, the given computing device can receive requests to de-identify protected data appearing in input text, and use the trained neural network to generate a version of the input text with de-identified protected data.

In some examples, two or more computing devices can be used to provide output predictions; e.g., a first computing device can generate and send requests to de-identify protected data appearing in input text. Then, the second computing device can use the trained versions of neural networks, perhaps after training, to generate a version of the input text with de-identified protected data, and respond to the requests from the first computing device. Then, upon reception of responses to the requests, the first computing device can provide the requested output (e.g., using a user interface and/or a display, a printed copy, an electronic communication, etc.).

### Example Data Network

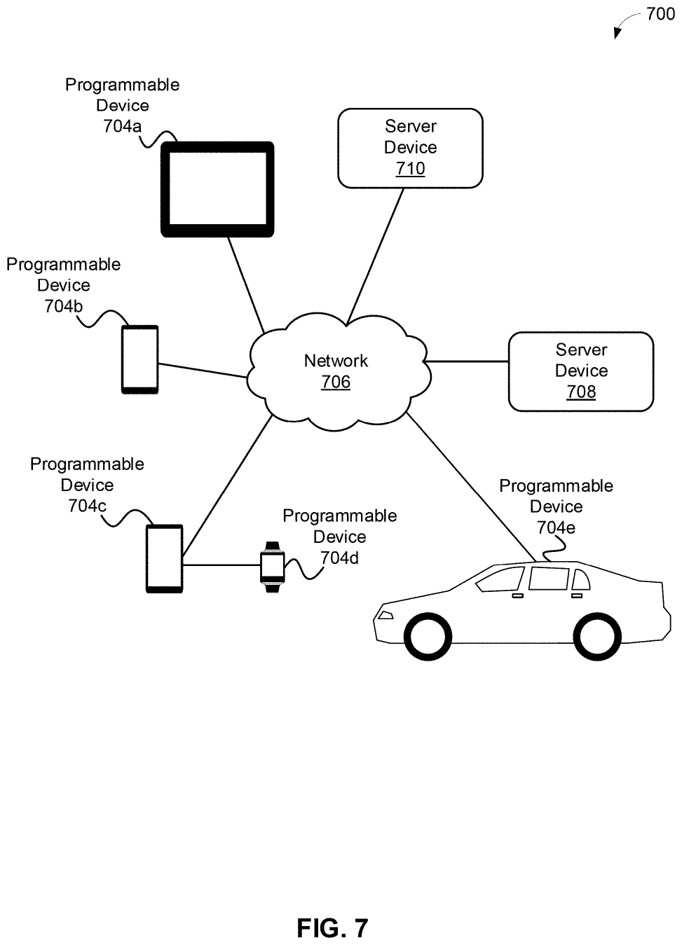

FIG. 7 depicts a distributed computing architecture 700, in accordance with example embodiments. Distributed computing architecture 700 includes server devices 708, 710 that are configured to communicate, via network 706, with programmable devices 704a, 704b, 704c, 704d, 704e. Network 706 may correspond to a local area network (LAN), a wide area network (WAN), a WLAN, a WWAN, a corporate intranet, the public Internet, or any other type of network configured to provide a communications path between networked computing devices. Network 706 may also correspond to a combination of one or more LANs, WANs, corporate intranets, and/or the public Internet.

Although FIG. 7 only shows five programmable devices, distributed application architectures may serve tens, hundreds, or thousands of programmable devices. Moreover, programmable devices 704a, 704b, 704c, 704d, 704e (or any additional programmable devices) may be any sort of computing device, such as a mobile computing device, desktop computer, wearable computing device, head-mountable device (HMD), network terminal, a mobile computing device, and so on. In some examples, such as illustrated by programmable devices 704a, 704b, 704c, 704e, programmable devices can be directly connected to network 706. In other examples, such as illustrated by programmable device 704d, programmable devices can be indirectly connected to network 706 via an associated computing device, such as programmable device 704c. In this example, programmable device 704c can act as an associated computing device to pass electronic communications between programmable device 704d and network 706. In other examples, such as illustrated by programmable device 704e, a computing device can be part of and/or inside a vehicle, such as a car, a truck, a bus, a boat or ship, an airplane, etc. In other examples not shown in FIG. 7, a programmable device can be both directly and indirectly connected to network 706.

Server devices 708, 710 can be configured to perform one or more services, as requested by programmable devices 704a-704e. For example, server device 708 and/or 710 can provide content to programmable devices 704a-704e. The content can include, but is not limited to, web pages, hypertext, scripts, binary data such as compiled software, images, audio, and/or video. The content can include compressed and/or uncompressed content. The content can be encrypted and/or unencrypted. Other types of content are possible as well.

As another example, server device 708 and/or 710 can provide programmable devices 704a-704e with access to software for database, search, computation, graphical, audio, video, World Wide Web/Internet utilization, and/or other functions. Many other examples of server devices are possible as well.

### Computing Device Architecture

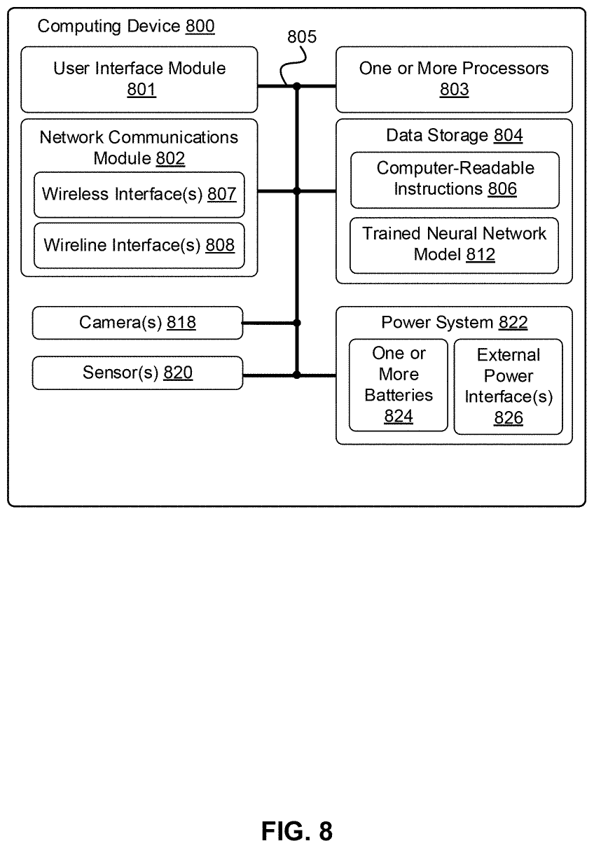

FIG. 8 is a block diagram of an example computing device 800, in accordance with example embodiments. In particular, computing device 800 shown in FIG. 8 can be configured to perform at least one function of and/or related to neural networks 200, 300, 400, 500, and/or method 1000.

Computing device 800 may include a user interface module 801, a network communications module 802, one or more processors 803, data storage 804, one or more cameras 818, one or more sensors 820, and power system 822, all of which may be linked together via a system bus, network, or other connection mechanism 805.

User interface module 801 can be operable to send data to and/or receive data from external user input/output devices. For example, user interface module 801 can be configured to send and/or receive data to and/or from user input devices such as a touch screen, a computer mouse, a keyboard, a keypad, a touch pad, a track ball, a joystick, a voice recognition module, and/or other similar devices. User interface module 801 can also be configured to provide output to user display devices, such as one or more cathode ray tubes (CRT), liquid crystal displays, light emitting diodes (LEDs), displays using digital light processing (DLP) technology, printers, light bulbs, and/or other similar devices, either now known or later developed. User interface module 801 can also be configured to generate audible outputs, with devices such as a speaker, speaker jack, audio output port, audio output device, earphones, and/or other similar devices. User interface module 801 can further be configured with one or more haptic devices that can generate haptic outputs, such as vibrations and/or other outputs detectable by touch and/or physical contact with computing device 800. In some examples, user interface module 801 can be used to provide a graphical user interface (GUI) for utilizing computing device 800. For example, user interface module 801 can be used to provide a version of the input text with de-identified protected data. Also, for example, user interface module 801 can be used to receive user input of text with potentially protected data to be de-identified.

Network communications module 802 can include one or more devices that provide one or more wireless interfaces 807 and/or one or more wireline interfaces 808 that are configurable to communicate via a network. Wireless interface(s) 807 can include one or more wireless transmitters, receivers, and/or transceivers, such as a Bluetooth™ transceiver, a Zigbee® transceiver, a Wi-Fi™ transceiver, a WiMAX™ transceiver, an LTE™ transceiver, and/or other type of wireless transceiver configurable to communicate via a wireless network. Wireline interface(s) 808 can include one or more wireline transmitters, receivers, and/or transceivers, such as an Ethernet transceiver, a Universal Serial Bus (USB) transceiver, or similar transceiver configurable to communicate via a twisted pair wire, a coaxial cable, a fiber-optic link, or a similar physical connection to a wireline network.

In some examples, network communications module 802 can be configured to provide reliable, secured, and/or authenticated communications. For each communication described herein, information for facilitating reliable communications (e.g., guaranteed message delivery) can be provided, perhaps as part of a message header and/or footer (e.g., packet/message sequencing information, encapsulation headers and/or footers, size/time information, and transmission verification information such as cyclic redundancy check (CRC) and/or parity check values). Communications can be made secure (e.g., be encoded or encrypted) and/or decrypted/decoded using one or more cryptographic protocols and/or algorithms, such as, but not limited to, Data Encryption Standard (DES), Advanced Encryption Standard (AES), a Rivest-Shamir-Adelman (RSA) algorithm, a Diffie-Hellman algorithm, a secure sockets protocol such as Secure Sockets Layer (SSL) or Transport Layer Security (TLS), and/or Digital Signature Algorithm (DSA). Other cryptographic protocols and/or algorithms can be used as well or in addition to those listed herein to secure (and then decrypt/decode) communications.

One or more processors 803 can include one or more general purpose processors, and/or one or more special purpose processors (e.g., digital signal processors, tensor processing units (TPUs), graphics processing units (GPUs), application specific integrated circuits, etc.). One or more processors 803 can be configured to execute computer-readable instructions 806 that are contained in data storage 804 and/or other instructions as described herein.

Data storage 804 can include one or more non-transitory computer-readable storage media that can be read and/or accessed by at least one of one or more processors 803. The one or more computer-readable storage media can include volatile and/or non-volatile storage components, such as optical, magnetic, organic or other memory or disc storage, which can be integrated in whole or in part with at least one of one or more processors 803. In some examples, data storage 804 can be implemented using a single physical device (e.g., one optical, magnetic, organic or other memory or disc storage unit), while in other examples, data storage 804 can be implemented using two or more physical devices.

Data storage 804 can include computer-readable instructions 806 and perhaps additional data. In some examples, data storage 804 can include storage required to perform at least part of the herein-described methods, scenarios, and techniques and/or at least part of the functionality of the herein-described devices and networks. In some examples, data storage 804 can include storage for a trained neural network model 812 (e.g., a model of trained neural network 100). In particular of these examples, computer-readable instructions 806 can include instructions that, when executed by processor(s) 803, enable computing device 800 to provide for some or all of the functionality of trained neural network model 812.

In some examples, computing device 800 can include one or more cameras 818. Camera(s) 818 can include one or more image capture devices, such as still and/or video cameras, equipped to capture videos. The one or more images can be one or more images utilized in video imagery. Camera(s) 818 can capture light and/or electromagnetic radiation emitted as visible light, infrared radiation, ultraviolet light, and/or as one or more other frequencies of light.

In some examples, computing device 800 can include one or more sensors 820. Sensors 820 can be configured to measure conditions within computing device 800 and/or conditions in an environment of computing device 800 and provide data about these conditions. For example, sensors 820 can include one or more of: (i) sensors for obtaining data about computing device 800, such as, but not limited to, a thermometer for measuring a temperature of computing device 800, a battery sensor for measuring power of one or more batteries of power system 822, and/or other sensors measuring conditions of computing device 800; (ii) an identification sensor to identify other objects and/or devices, such as, but not limited to, a Radio Frequency Identification (RFID) reader, proximity sensor, one-dimensional barcode reader, two-dimensional barcode (e.g., Quick Response (QR) code) reader, and a laser tracker, where the identification sensors can be configured to read identifiers, such as RFID tags, barcodes, QR codes, and/or other devices and/or object configured to be read and provide at least identifying information; (iii) sensors to measure locations and/or movements of computing device 800, such as, but not limited to, a tilt sensor, a gyroscope, an accelerometer, a Doppler sensor, a GPS device, a sonar sensor, a radar device, a laser-displacement sensor, and a compass; (iv) an environmental sensor to obtain data indicative of an environment of computing device 800, such as, but not limited to, an infrared sensor, an optical sensor, a light sensor, a biosensor, a capacitive sensor, a touch sensor, a temperature sensor, a wireless sensor, a radio sensor, a movement sensor, a microphone, a sound sensor, an ultrasound sensor and/or a smoke sensor; and/or (v) a force sensor to measure one or more forces (e.g., inertial forces and/or G-forces) acting about computing device 800, such as, but not limited to one or more sensors that measure: forces in one or more dimensions, torque, ground force, friction, and/or a zero moment point (ZMP) sensor that identifies ZMPs and/or locations of the ZMPs. Many other examples of sensors 820 are possible as well.

Power system 822 can include one or more batteries 824 and/or one or more external power interfaces 826 for providing electrical power to computing device 800. Each battery of the one or more batteries 824 can, when electrically coupled to the computing device 800, act as a source of stored electrical power for computing device 800. One or more batteries 824 of power system 822 can be configured to be portable. Some or all of one or more batteries 824 can be readily removable from computing device 800. In other examples, some or all of one or more batteries 824 can be internal to computing device 800, and so may not be readily removable from computing device 800. Some or all of one or more batteries 824 can be rechargeable. For example, a rechargeable battery can be recharged via a wired connection between the battery and another power supply, such as by one or more power supplies that are external to computing device 800 and connected to computing device 800 via the one or more external power interfaces. In other examples, some or all of one or more batteries 824 can be non-rechargeable batteries.

One or more external power interfaces 826 of power system 822 can include one or more wired-power interfaces, such as a USB cable and/or a power cord, that enable wired electrical power connections to one or more power supplies that are external to computing device 800. One or more external power interfaces 826 can include one or more wireless power interfaces, such as a Qi wireless charger, that enable wireless electrical power connections, such as via a Qi wireless charger, to one or more external power supplies. Once an electrical power connection is established to an external power source using one or more external power interfaces 826, computing device 800 can draw electrical power from the external power source the established electrical power connection. In some examples, power system 822 can include related sensors, such as battery sensors associated with the one or more batteries or other types of electrical power sensors.

### Cloud-Based Servers

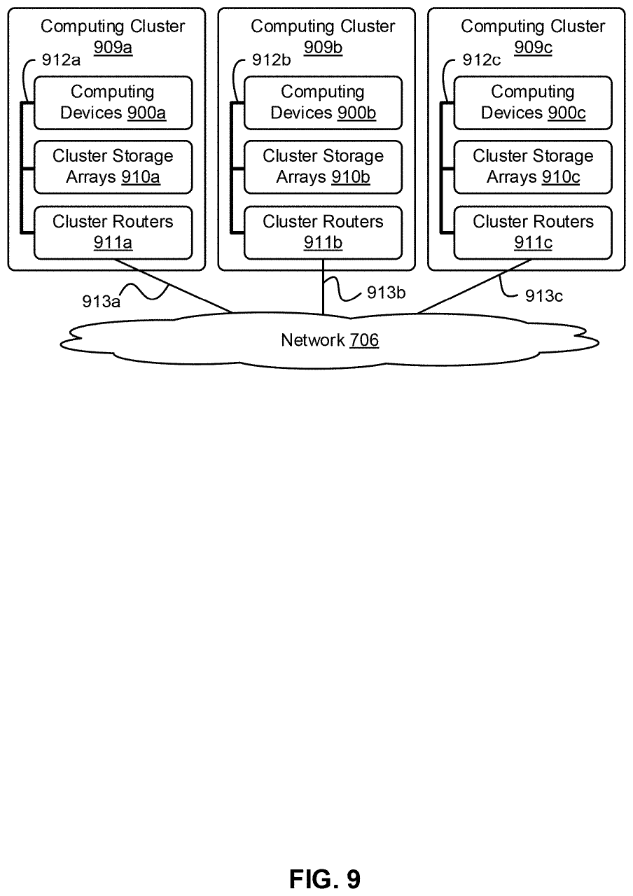

FIG. 9 depicts a network 706 of computing clusters 909a, 909b, 909c arranged as a cloud-based server system in accordance with an example embodiment. Computing clusters 909a, 909b, and 909c can be cloud-based devices that store program logic and/or data of cloud-based applications and/or services; e.g., perform at least one function of and/or related to neural network 100, and/or method 1000.

In some embodiments, computing clusters 909a, 909b, and 909c can be a single computing device residing in a single computing center. In other embodiments, computing clusters 909a, 909b, and 909c can include multiple computing devices in a single computing center, or even multiple computing devices located in multiple computing centers located in diverse geographic locations. For example, FIG. 9 depicts each of computing clusters 909a, 909b, and 909c residing in different physical locations.

In some embodiments, data and services at computing clusters 909a, 909b, 909c can be encoded as computer readable information stored in non-transitory, tangible computer readable media (or computer readable storage media) and accessible by other computing devices. In some embodiments, computing clusters 909a, 909b, 909c can be stored on a single disk drive or other tangible storage media, or can be implemented on multiple disk drives or other tangible storage media located at one or more diverse geographic locations.

FIG. 9 depicts a cloud-based server system in accordance with an example embodiment. In FIG. 9, functionality of neural network 100, and/or a computing device can be distributed among computing clusters 909a, 909b, and 909c. Computing cluster 909a can include one or more computing devices 900a, cluster storage arrays 910a, and cluster routers 911a connected by a local cluster network 912a. Similarly, computing cluster 909b can include one or more computing devices 900b, cluster storage arrays 910b, and cluster routers 911b connected by a local cluster network 912b. Likewise, computing cluster 909c can include one or more computing devices 900c, cluster storage arrays 910c, and cluster routers 911c connected by a local cluster network 912c.

In some embodiments, each of computing clusters 909a, 909b, and 909c can have an equal number of computing devices, an equal number of cluster storage arrays, and an equal number of cluster routers. In other embodiments, however, each computing cluster can have different numbers of computing devices, different numbers of cluster storage arrays, and different numbers of cluster routers. The number of computing devices, cluster storage arrays, and cluster routers in each computing cluster can depend on the computing task or tasks assigned to each computing cluster.

In computing cluster 909a, for example, computing devices 900a can be configured to perform various computing tasks of a neural network, an audio separation network, an audio embedding network, a video embedding network, a classifier, and/or a computing device. In one embodiment, the various functionalities of a neural network, an audio separation network, an audio embedding network, a video embedding network, a classifier, and/or a computing device can be distributed among one or more of computing devices 900a, 900b, and 900c. Computing devices 900b and 900c in respective computing clusters 909b and 909c can be configured similarly to computing devices 900a in computing cluster 909a. On the other hand, in some embodiments, computing devices 900a, 900b, and 900c can be configured to perform different functions.

In some embodiments, computing tasks and stored data associated with a neural network, a tokenizer, a token normalization block, a pre-trained token embedding model, a bi-directional recurrent neural network (BiRNN), a casing feature, a named entity sequence tagger, a tag prediction layer, a conditional random field, and/or a computing device can be distributed across computing devices 900a, 900b, and 900c based at least in part on the processing requirements of a neural network, a tokenizer, a token normalization block, a pre-trained token embedding model, a BiRNN, a casing feature, a named entity sequence tagger, a tag prediction layer, a conditional random field, and/or a computing device, the processing capabilities of computing devices 900a, 900b, 900c, the latency of the network links between the computing devices in each computing cluster and between the computing clusters themselves, and/or other factors that can contribute to the cost, speed, fault-tolerance, resiliency, efficiency, and/or other design goals of the overall system architecture.

Cluster storage arrays 910a, 910b, 910c of computing clusters 909a, 909b, and 909c can be data storage arrays that include disk array controllers configured to manage read and write access to groups of hard disk drives. The disk array controllers, alone or in conjunction with their respective computing devices, can also be configured to manage backup or redundant copies of the data stored in the cluster storage arrays to protect against disk drive or other cluster storage array failures and/or network failures that prevent one or more computing devices from accessing one or more cluster storage arrays.

Similar to the manner in which the functions of a neural network, a tokenizer, a token normalization block, a pre-trained token embedding model, a BiRNN, a casing feature, a named entity sequence tagger, a tag prediction layer, a conditional random field, and/or a computing device can be distributed across computing devices 900a, 900b, 900c of computing clusters 909a, 909b, 909c, various active portions and/or backup portions of these components can be distributed across cluster storage arrays 910a, 910b, 910c. For example, some cluster storage arrays can be configured to store one portion of the data of a neural network, a tokenizer, a token normalization block, a pre-trained token embedding model, a BiRNN, a casing feature, a named entity sequence tagger, a tag prediction layer, a conditional random field, and/or a computing device, while other cluster storage arrays can store other portion(s) of data of a neural network, a tokenizer, a token normalization block, a pre-trained token embedding model, a BiRNN, a casing feature, a named entity sequence tagger, a tag prediction layer, a conditional random field, and/or a computing device. Also, for example, some cluster storage arrays can be configured to store the data of a first neural network, while other cluster storage arrays can store the data of a second and/or third neural network. Additionally, some cluster storage arrays can be configured to store backup versions of data stored in other cluster storage arrays.

Cluster routers 911a, 911b, 911c in computing clusters 909a, 909b, and 909c can include networking equipment configured to provide internal and external communications for the computing clusters. For example, cluster routers 911a in computing cluster 909a can include one or more internet switching and routing devices configured to provide (i) local area network communications between computing devices 900a and cluster storage arrays 910a via local cluster network 912a, and (ii) wide area network communications between computing cluster 909a and computing clusters 909b and 909c via wide area network link 913a to network 706. Cluster routers 911b and 911c can include network equipment similar to cluster routers 911a, and cluster routers 911b and 911c can perform similar networking functions for computing clusters 909b and 909b that cluster routers 911a perform for computing cluster 909.

In some embodiments, the configuration of cluster routers 911a, 911b, 911c can be based at least in part on the data communication requirements of the computing devices and cluster storage arrays, the data communications capabilities of the network equipment in cluster routers 911a, 911b, 911c, the latency and throughput of local cluster networks 912a, 912b, 912c, the latency, throughput, and cost of wide area network links 913a, 913b, 913c, and/or other factors that can contribute to the cost, speed, fault-tolerance, resiliency, efficiency and/or other design criteria of the moderation system architecture.

### Example Methods of Operation

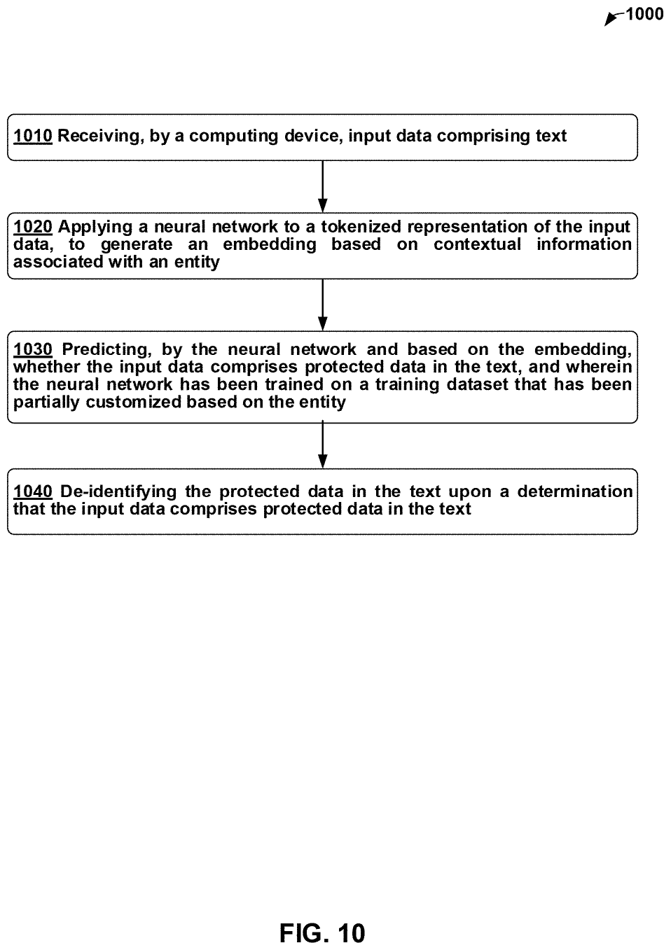

FIG. 10 is a flowchart of a method 1000, in accordance with example embodiments. Method 1000 can be executed by a computing device, such as computing device 800. Method 1000 can begin at block 1010, where the computing device can receive input data comprising text.

At block 1020, the method can involve applying a neural network to a tokenized representation of the input text, to generate an embedding based on contextual information associated with an entity.

At block 1030, the method can involve predicting, by the neural network and based on the embedding, whether the input data comprises protected data in the text, wherein the neural network has been trained on a training dataset that has been partially customized based on the entity.

At block 1040, the method can involve de-identifying the protected data in the text upon a determination that the input data includes protected data in the text.

In some embodiments, the training dataset that has been partially customized based on the entity may include unlabeled training data.

In some embodiments, the method can involve training the neural network to receive a particular input data comprising particular text, and predict whether the particular input data comprises protected data in the particular text.

In some embodiments, the training of the neural network may include training the neural network based on a mixture of (i) labeled data from the training dataset that has been partially customized based on the entity, and (ii) an unlabeled training dataset.

In some embodiments, the training of the neural network may include a pre-training of the neural network based on a first dataset comprising labeled training data. In such embodiments, the method can further involve a training of the pre-trained neural network based on labeled data from the training dataset that has been partially customized based on the entity.

In some embodiments, the method can involve providing a platform to generate a manually programmed dictionary of terms indicative of protected data. In such embodiments, the method can further involve receiving at least a portion of the training dataset that has been partially customized based on the entity from the platform. In some embodiments, the providing of the platform can include providing an applications programming interface (API).

In some embodiments, the method can involve obtaining a first dataset comprising labeled training data that includes first protected data, where the labeled training data is not based on the entity. Then, the method can involve obtaining a second dataset comprising unlabeled training data that includes second protected data, where the unlabeled training data is based on the entity. The methods may also involve generating, based on the second dataset, a customized token embedding of the second protected data. Then, the method can involve applying the customized token embedding to the first dataset. The training of the neural network can include training the neural network on the first dataset based on the customized token embedding. In some embodiments, the customized token embedding may be based on an algorithm to estimate word representations in a vector space.

In some embodiments, the neural network can include a pre-trained token embedding model to map the tokenized representation to a first multidimensional vector space. The neural network can also include a BiRNN to generate a character-based token embedding that maps each token of the one or more tokens to a second multidimensional vector space. Additionally, the neural network can include a second BiRNN to add contextual information to the tokenized representation. The neural network can also include a prediction layer to project the first multidimensional vector space onto a probability distribution over a plurality of tags, wherein the plurality of tags are indicative of the protected data. In some embodiments, the neural network can include a second prediction layer based on a conditional random field, wherein the conditional random field determines whether a tag of the plurality of tags is consistent as a sequence.

In some embodiments, the method can involve generating the tokenized representation by generating one or more tokens based on the input text. Then, the method can involve, for each token of the one or more tokens, converting (i) a character to a lowercase letter, and (ii) a numeral to zero.

In some embodiments, the protected data may include protected health information. The training dataset that has been partially customized based on the entity may include radiology images that include the protected health information.

In some embodiments, the training dataset that has been partially customized based on the entity may include free text unstructured notes. In some embodiments, the free text unstructured notes may include free text notes associated with a discharge of a patient.

In some embodiments, the method can involve determining, by the computing device, a request to de-identify potentially protected data in a given input data. Then, the method can involve sending the request to de-identify the potentially protected data from the computing device to a second computing device, the second computing device comprising a trained version of the neural network. After sending the request, the method can involve the computing device receiving, from the second computing device, the predicting of whether the given input data comprises protected data. Then, the method can involve de-identifying the protected data in the given input data.

In some embodiments, the method can involve obtaining a trained neural network at the computing device. The predicting of whether the input data comprises protected data in the text may include predicting by the computing device using the trained neural network.

In some embodiments, the protected data may include one or more of personally identifiable information (PII), protected health information (PHI), or payment card industry (PCI) information.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims.

The above detailed description describes various features and functions of the disclosed systems, devices, and methods with reference to the accompanying figures. In the figures, similar symbols typically identify similar components, unless context dictates otherwise. The illustrative embodiments described in the detailed description, figures, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the ladder diagrams, scenarios, and flow charts in the figures and as discussed herein, each block and/or communication may represent a processing of information and/or a transmission of information in accordance with example embodiments. Alternative embodiments are included within the scope of these example embodiments. In these alternative embodiments, for example, functions described as blocks, transmissions, communications, requests, responses, and/or messages may be executed out of order from that shown or discussed, including substantially concurrent or in reverse order, depending on the functionality involved. Further, more or fewer blocks and/or functions may be used with any of the ladder diagrams, scenarios, and flow charts discussed herein, and these ladder diagrams, scenarios, and flow charts may be combined with one another, in part or in whole.

A block that represents a processing of information may correspond to circuitry that can be configured to perform the specific logical functions of a herein-described method or technique. Alternatively or additionally, a block that represents a processing of information may correspond to a module, a segment, or a portion of program code (including related data). The program code may include one or more instructions executable by a processor for implementing specific logical functions or actions in the method or technique. The program code and/or related data may be stored on any type of computer readable medium such as a storage device including a disk or hard drive or other storage medium.

The computer readable medium may also include non-transitory computer readable media such as non-transitory computer-readable media that stores data for short periods of time like register memory, processor cache, and random access memory (RAM). The computer readable media may also include non-transitory computer readable media that stores program code and/or data for longer periods of time, such as secondary or persistent long term storage, like read only memory (ROM), optical or magnetic disks, compact-disc read only memory (CD-ROM), for example. The computer readable media may also be any other volatile or non-volatile storage systems. A computer readable medium may be considered a computer readable storage medium, for example, or a tangible storage device.

Moreover, a block that represents one or more information transmissions may correspond to information transmissions between software and/or hardware modules in the same physical device. However, other information transmissions may be between software modules and/or hardware modules in different physical devices.

While various aspects and embodiments have been disclosed herein, other aspects and embodiments will be apparent to those skilled in the art. The various aspects and embodiments disclosed herein are provided for explanatory purposes and are not intended to be limiting, with the true scope being indicated by the following claims.

What is claimed is:

1. A computer-implemented method, comprising:
receiving, by a computing device, input data comprising text;
applying a neural network to a tokenized representation of the input data, to generate an embedding based on contextual information associated with an entity;
predicting, by the neural network and based on the embedding, whether the input data comprises protected data in the text, and wherein the neural network has been trained on a training dataset that has been partially customized based on the entity; and
upon a determination that the input data comprises protected data in the text, de-identifying the protected data in the text.

2. The computer-implemented method of claim 1, wherein the training dataset that has been partially customized based on the entity comprises unlabeled training data.

3. The computer-implemented method of claim 1, further comprising:
training the neural network to receive a particular input data comprising particular text, and predict whether the particular input data comprises protected data in the particular text.

4. The computer-implemented method of claim 3, wherein the training of the neural network comprises training the neural network based on a mixture of (i) labeled data from the training dataset that has been partially customized based on the entity, and (ii) an unlabeled training dataset.

5. The computer-implemented method of claim 3, wherein the training of the neural network comprises:
a pre-training of the neural network based on a first dataset comprising labeled training data; and
a training of the pre-trained neural network based on labeled data from the training dataset that has been partially customized based on the entity.

6. The computer-implemented method of claim 3, further comprising:
providing a platform to generate a manually programmed dictionary of terms indicative of protected data; and
receiving at least a portion of the training dataset that has been partially customized based on the entity from the platform.

7. The computer-implemented method of claim 6, wherein the providing of the platform comprises providing an applications programming interface (API).

8. The computer-implemented method of claim 3, further comprising:
obtaining a first dataset comprising labeled training data that includes first protected data, wherein the labeled training data is not based on the entity;
obtaining a second dataset comprising unlabeled training data that includes second protected data, wherein the unlabeled training data is based on the entity;
generating, based on the second dataset, a customized token embedding of the second protected data;
applying the customized token embedding to the first dataset, and
wherein the training of the neural network comprises training the neural network on the first dataset based on the customized token embedding.

9. The computer-implemented method of claim 8, wherein the customized token embedding is based on an algorithm to estimate word representations in a vector space.

10. The computer-implemented method of claim 1, wherein the neural network comprises:
a pre-trained token embedding model to map the tokenized representation to a first multidimensional vector space;
a bi-directional recurrent neural network (BiRNN) to generate a character-based token embedding that maps each token of the one or more tokens to a second multidimensional vector space; and
a second BiRNN to add contextual information to the tokenized representation;
a prediction layer to project the first multidimensional vector space onto a probability distribution over a plurality of tags, wherein the plurality of tags are indicative of the protected data.

11. The computer-implemented method of claim 10, wherein the neural network comprises:
a second prediction layer based on a conditional random field, wherein the conditional random field determines whether a tag of the plurality of tags is consistent as a sequence.

12. The computer-implemented method of claim 1, further comprising:
generating the tokenized representation by generating one or more tokens based on the input text; and
for each token of the one or more tokens, converting (i) a character to a lowercase letter, and (ii) a numeral to zero.

13. The computer-implemented method of claim 1, wherein the protected data comprises protected health information, and wherein the training dataset that has been partially customized based on the entity comprises radiology images that include the protected health information.

14. The computer-implemented method of claim 1, wherein the training dataset that has been partially customized based on the entity comprises free text unstructured notes.

15. The computer-implemented method of claim 14, wherein the free text unstructured notes comprise free text notes associated with a discharge of a patient.

16. The computer-implemented method of claim 1, further comprising:
determining, by the computing device, a request to de-identify potentially protected data in a given input data;
sending the request to de-identify the potentially protected data from the computing device to a second computing device, the second computing device comprising a trained version of the neural network;
after sending the request, the computing device receiving, from the second computing device, the predicting of whether the given input data comprises protected data; and
de-identifying the protected data in the given input data.

17. The computer-implemented method of claim 1, further comprising:
obtaining a trained neural network at the computing device, and
wherein the predicting of whether the input data comprises protected data in the text comprises predicting by the computing device using the trained neural network.

18. The computer-implemented method of claim 1, wherein the protected data comprises one or more of personally identifiable information (PII), protected health information (PHI), or payment card industry (PCI) information.

19. A server for de-identifying data, comprising:
one or more processors; and
memory storing computer-executable instructions that, when executed by the one or more processors, cause the server to perform operations comprising:
receiving input data comprising text;
applying a neural network to a tokenized representation of the input text, to generate an embedding based on contextual information associated with an entity;
predicting, by the neural network and based on the embedding, whether the input data comprises protected data in the text, wherein the neural network has been trained on a training dataset that has been partially customized based on the entity; and
upon a determination that the input data comprises protected data in the text, de-identifying the protected data in the text.

20. An article of manufacture comprising one or more computer readable media having computer-readable instructions stored thereon that, when executed by one or more processors of a computing device, cause the computing device to carry out operations comprising:
receiving, by the computing device, input data comprising text;
applying a neural network to a tokenized representation of the input text, to generate an embedding based on contextual information associated with an entity;
predicting, by the neural network and based on the embedding, whether the input data comprises protected data in the text, and wherein the neural network has been trained on a training dataset that has been partially customized based on the entity; and
upon a determination that the input data comprises protected data in the text, de-identifying the protected data in the text.
